(19) United States
(12) Patent Application Publication
(10) Pub. No.: US 2005/0086007 A1
(43) Pub. Date: Apr. 21, 2005

(54) Method and apparatus for computer assisted analysis of compounds

(76) Inventors: J. Barry Shackleford, Portola Valley, CA (US); Motoo Tanaka, Tokyo (JP)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/688,249

(22) Filed: Oct. 16, 2003

(51) Int. Cl. G06F 19/00; G01N 33/48; G01N 33/50

(52) U.S. Cl. 702/19

(57) ABSTRACT

A method and apparatus is used to discover elements in a compound with a fitness function by receiving a set of monoisotopic mass look-up-tables (LUTs), identifying mass values in parallel by cross-referencing monoisotopic mass LUT addresses, evaluating different permutations of the identified mass values, accessing values in mass spectroscopy data sets according to the permutations of mass values and determining the combination of elements in the compound according to a correlation between the permutations of mass values and the mass values associated with the mass spectroscopy data set. Performing discovery with genetic algorithms includes receiving a population of electronic chromosomes having a fitness value, selecting electronic chromosomes randomly from the population of electronic chromosomes as parent electronic chromosomes, creating child electronic chromosome through a crossover of the parent electronic chromosomes and evaluating the fitness value of the child electronic chromosome to determine if the elements correspond to the compound.

### BACKGROUND OF THE INVENTION

The present invention relates to determining the elemental composition of compounds and materials using data processing.

An increasing number of pharmaceutical and drug companies focus their research on determining the elements that make up various compounds and materials. Determining the elemental composition of compounds is an important part of research in areas like proteomics, combinatorial chemistry, genetics, medicinal research, high-throughput screening of potential medicinal drugs and many other areas. The traditional methods of discovery required tedious laboratory work and manual labor. Many of the calculations and estimations are made manually or by specialized computer programs.

Clearly, the increased use of computers has paved the way to more rapid research as new research results can be quickly entered into a computer and analyzed. Harnessing the power of computer systems and the software programs to analyze the information improves both the speed and accuracy of identifying certain substances or their composition. To ensure that a solution is not missed, these programs often test almost all the potential combinations taking a more “brute force” approach to analysis. Fortunately, more powerful computer system designs can keep up with a majority of the analysis and complete the research even it requires computations lasting days, weeks and sometimes years at time.

Unfortunately, these brute force computational schemes and powerful computers eventually will not satisfy research times required for modern drug discovery. A growing number of researchers would like to further reduce the time period required for analyzing compounds and their composition by several orders of magnitude. Rapid analysis and discover is becoming more important as the need to identify and treat diseases more rapidly and with fewer resources is upon these researchers. As a result, more efficient computational approaches to drug-discovery and analysis are needed to help keep up with current research trends.

### BRIEF DESCRIPTION OF THE DRAWINGS

FIG. 1A depicts a flowchart diagram of the operations for analyzing a sample using a mass spectrometer;

FIG. 1B is a hypothetical and schematic plot of the monoisotopic peaks associated with ions produced from a sample processed through a mass spectroscopy device;

FIG. 2 is a table listing a set of amino acids for a protein sequencing problem;

FIG. 3 is a flow chart diagram of the operations for performing genetic algorithm (GA) analysis in accordance with one implementation of the present invention;

FIG. 4 is a block diagram illustrating both a cross-over operation between parent chromosomes and a mutation operation on a child chromosome;

FIG. 5 is block diagram representation of a GA circuit 500 designed in accordance with one implementation of the present invention;

FIG. 6 is a flowchart diagram of the operations for using a fitness function in accordance with the present invention;

FIG. 7 is a block diagram illustration of a fitness function implemented in hardware and used to identify an amino acid sequence for a protein; and

FIG. 8 is a block diagram of a system used in one implementation for performing the apparatus or methods of the present invention.

Like reference numbers and designations in the various drawings indicate like elements.

### SUMMARY OF THE INVENTION

One aspect of the present invention features a method of discovering the elements in a compound with a fitness function. The discovery of elements includes receiving a set of monoisotopic mass look-up-tables (LUTs) that associates LUT addresses with mass values for a set of elements, identifying mass values in parallel by cross-referencing two or more LUT addresses associated with an electronic chromosome and addresses in the monoisotopic mass LUTs, evaluating different permutations of the identified mass values from the set of monoisotopic mass LUTs, accessing values in two or more mass spectroscopy data sets according to the permutations of mass values identified in the monoisotopic mass LUTs and determining the combination of elements in the compound according to a correlation between the permutations of mass values and the mass values associated with the mass spectroscopy data set.

Another aspect of the present invention describes a method of discovering elements of a compound using genetic algorithm computations. Discovering compounds with genetic algorithms includes receiving a population of electronic chromosomes each representing a set of elements and having a fitness value determined using a fitness function indicating the probability that at least one of the electronic chromosome provides the elements in the compound, selecting two electronic chromosomes randomly from the population of electronic chromosomes as parent electronic chromosomes, creating a child electronic chromosome through a crossover of the two parent electronic chromosomes using one or more randomly selected cut-points on the parent electronic chromosomes and evaluating the fitness value of the resulting child electronic chromosome using the fitness function to determine if elements in the child electronic chromosome correspond to the compound.

### DETAILED DESCRIPTION

Aspects of the present invention are advantageous in at least one or more of the following ways. Implementations of the present invention determine the elements of a compound or material more rapidly by application of a fitness function. The fitness function gives a figure-of-merit numeric value for a potential solution; the better the potential solution, the better the numeric value produced by the fitness function. Using the fitness as a guide, during the analysis helps identify and converge on an optional solution.

This is useful, for example, in proteomic research when determining the amino acids that make up a particular protein compound. The fitness function is applied to various combinations of amino acids producing a fitness value for the particular amino acid combination. This fitness function can be applied to a large number of sample combinations to determine the combination of amino acids most likely to make up the protein being studied. Using the fitness function of the present invention also provides an objective measurement for evaluating different combinations of elements in light of a protein or other compound being studied.

As a further advantage, a fitness function of the present invention reduces research time by evaluating multiple permutations of elements in parallel. Conventional methods in combinatorial chemistry and proteomic research perform similar aspects of analysis in serial; this increases laboratory and research times. Implementations of the present invention instead process different combinations of elements in parallel. The fitness function of the present invention investigates many operations in parallel during the same time period or interval. More sample combination of elements can be processed in a shorter time frame allowing more rapid discovery and reduced research costs.

Further advantages are realized by using a fitness function of the present invention along with a genetic analysis (GA) methodology and framework. In addition to the advantages described above, GA improves the likelihood of finding the combination of elements in a protein or other compound by increasing the sample or population of combinations being analyzed. An initial set of combinations for analysis are increased by cutting “parent” combinations and recombining them into a new “children” combinations of elements. Additional sample combinations of elements are made available by introducing a “mutation” or random variation in existing members of the sample set or population. The larger sample set or population increases the likelihoods of identifying a solution in proteomic and combinatorial chemistry research where there are many unknowns.

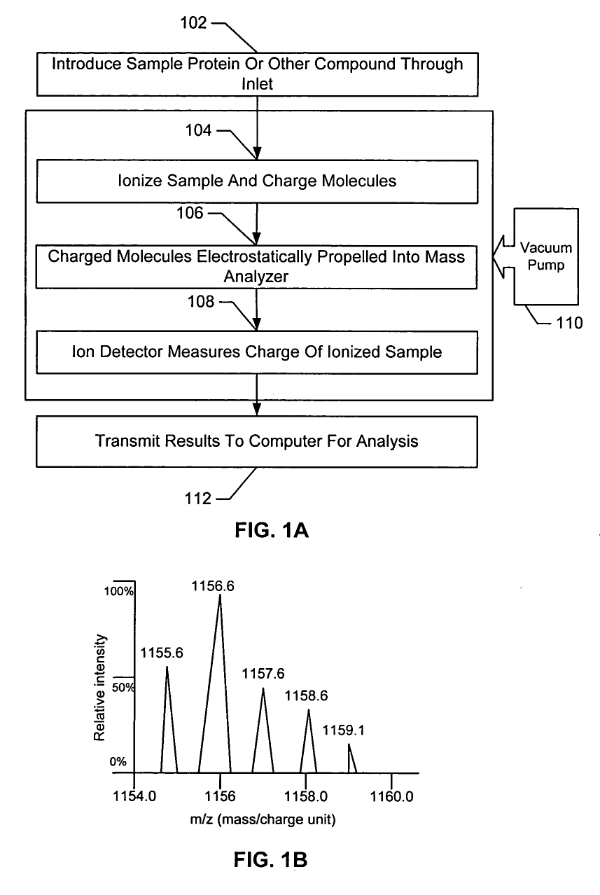

FIG. 1A depicts a flowchart diagram of the operations for analyzing a sample using a mass spectrometer. Initially, a sample is introduced through an inlet of the mass spectrometer (102). Sensitive and properly calibrated mass spectrometers can operate on extremely small samples of biological materials and perform highly accurate analysis. Scientist now regularly rely on mass spectroscopy to identify active genes in cells and proteins which conventional genetic methods cannot readily process.

An ion source ionizes the sample introduced and charges the molecules (104). With biological samples like proteins and genetic material, two ion-producing methods are typically used: electrospray ionization (ESI) and the matrix-assisted laser desorption ionization (MALDI). Both these methods are able to heat the samples into a gaseous state and charge them without destruction. In addition to ionization, chemicals or enzymes may also be introduced to further break the protein samples into peptide fragments.

While under vacuum by vacuum pump 110, the charged molecules are electrostatically propelled into mass analyzer (106) and ion detector that measure the charge of the ionized sample (108). The time of flight it takes a molecule to travel from the point of ionization to the detector is a function of the mass-to-charge (m/z) ratio of a particle. Large molecules with greater mass travel slower than smaller molecules while molecules with larger charge (e.g. 2+) tend to travel faster than molecules with a smaller charge (e.g. 1+). Results are transmitted to a computer for analysis (112) where the principles of chemistry, physics and other disciplines are used to analyze and identify the compound.

FIG. 1B is a hypothetical and schematic plot of the monoisotopic peaks associated with ions produced from a sample processed through a mass spectroscopy device. Each spike corresponds to different elements of a sample compound and the relative amounts each element occupies in the compound. Unknown compounds can be identified by narrowing the field of possible elements in the compound and then matching the mass-charge (m/z) measurements along with the relative intensity levels to determine both a sequence and quantity of elements in the compound. In the case of proteins, it is known that all proteins are created from a combination of 20 amino acids joined together by covalent bonds. These 20 amino acids are organized into polypeptides of varying length, different linear sequences, and three-dimensional configurations. While DNA sequences can be used to identify a protein, it is often more advantageous to classify and identify proteins directly. The mass spectrometer accurately measures the mass of a compound but careful analysis is still needed to interpret the results. Unfortunately, protein identification using conventional analysis remains time consuming and compute/resource intensive compared with implementations of the present invention. This is also true for analyzing data associated with combinatorial chemistry, medicinal chemistry and other similar areas of research having large sample sets and complex analytic requirements.

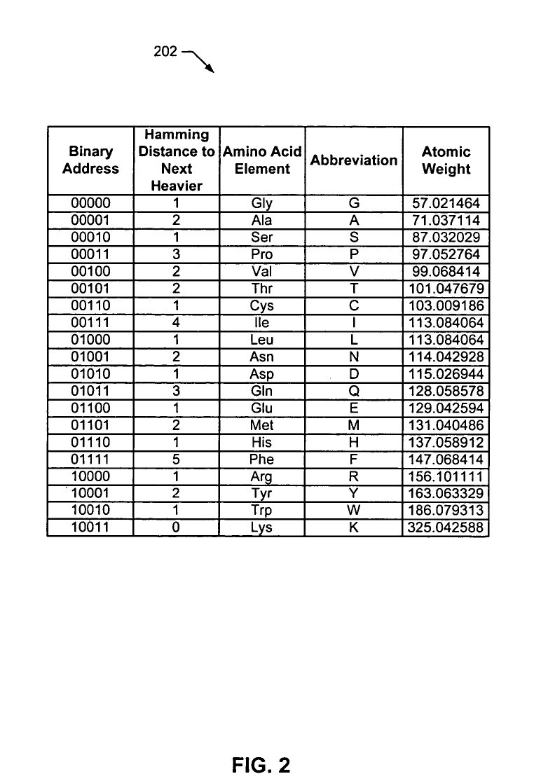

FIG. 2 is a table 202 listing a set of amino acids for a protein sequencing problem. Here, table 202 includes a binary address to identify the amino acid, a hamming distance to the next heavier amino acid, a short name (i.e., three letters) of each amino acid, an abbreviation of the amino acid (i.e., a single letter), and the corresponding atomic weight of each amino acid. A more advantageous organization of data in table 202 reduces the hamming distance between adjacent elements and is described in co-pending U.S. application Ser. No. 10/367,563 assigned to the assignee of the present invention entitled, “STORAGE METHOD AND APPARATUS FOR GENETIC ALGORITHM ANALYSIS” by Shackleford and Tanaka.

A GA system using table 202 arranges the binary numbering along with ascending/descending atomic weight of the respective amino acids. In table 202, the amino acids are arranged in increasing atomic weight and an increasing binary number sequence going from 0000₂ (“zero”) to 10011₂ (“nineteen”). In an alternate GA system, amino acids may be arranged alphabetically as well in other various orders using the same binary number sequence. One or more binary addresses in table 202 correspond to different amino acids and when combined together in subfields represent the electronic chromosome used in the GA analysis.

Mutation is an important computational mechanism for introducing different amino acids in the GA analysis that otherwise may not have been available directly from the sample set or population. In operation, these different amino acids are introduced by randomly changing bits in the binary address representation of the chromosome with a low probability. Each subfield portion of the binary address affected by the mutation specifies a different amino acid as the GA analysis of the present invention attempts to converge on a solution. Because single-bit mutations are more likely to occur, next heavier amino acids in conventional table 202 with a Hamming distance closest to “1” are more likely to be selected through the mutation process.

For example, a single-bit mutation is more likely to select the “Ala”, “Pro”, “Ile”, “Leu”, “Gln”, “Met”, “Phe”, “Tyr”, and “Lys” amino acids than the other next heavier amino acids in conventional table 202. Adjacent lighter elements from these amino acids are distinguished from other elements in conventional table 202 as they are separated by only a hamming distance of 1. In contrast, a mutation applied to a chromosome with a subfield representing “Phe” is as unlikely to result in selecting the next heavier amino acid “Arg” as the probability of producing a five-bit mutation is improbable. Consequently, a mutation using conventional table 202 favors the selection of certain amino acids due to the organization of data in conventional table 202 rather than the ability to provide an optimal solution. This tends to limit the scope of solutions being explored during GA analysis and potentially delay convergence upon a more optimal solution. As previously mentioned, the co-pending U.S. Patent Application entitled “STORAGE METHOD AND APPARATUS FOR GENETIC ALGORITHM ANALYSIS” addresses this scenario should it occur by rearranging entries in the table being analyzed.

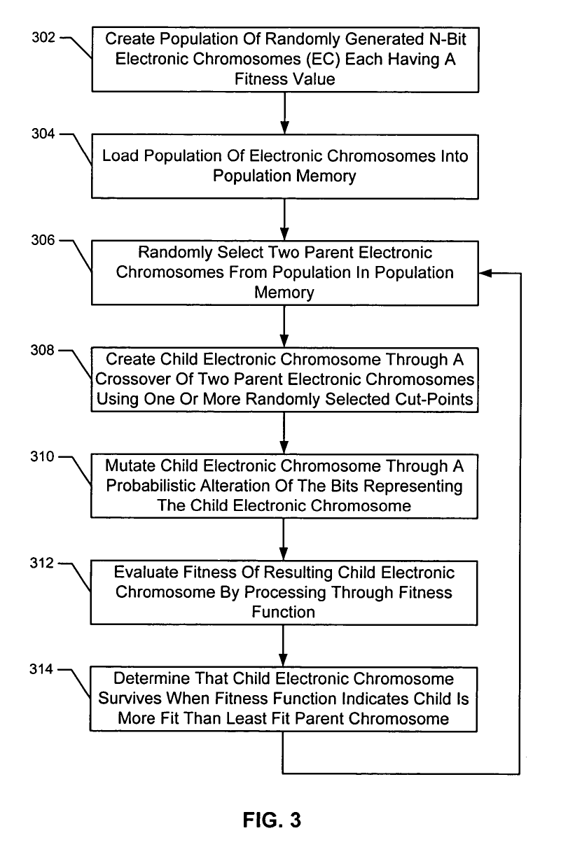

FIG. 3 is a flow chart diagram of the operations for performing genetic algorithm (GA) analysis in accordance with one implementation of the present invention. To begin GA analysis, a population of randomly generated n-bit electronic chromosomes (hereinafter referred to as chromosomes) is created and stored in population memory or other storage areas (302). Typically, the population memory also holds a fitness value corresponding each of the n-bit chromosomes in the population. Each chromosome is evaluated by a fitness function and assigned a fitness value based on how well the chromosome appears to solve the problem being analyzed. Moreover, the fitness value determines which chromosomes will be kept in population memory and, eventually, the chromosome that best solves the problem being analyzed. For example, in proteomics the fitness value indicates the degree in which a chromosome provides the amino acid combination found in the particular protein being analyzed or studied.

The population memory is loaded with random n-bit binary patterns representing the chromosomes and corresponding m-bit fitness values assigned to each chromosome and related to the problem being studied (304). Implementations of the present invention initially fill the population memory with high-quality random numbers using cellular automata. Details on random number generation using cellular automata are described in co-pending U.S. patent application Ser. No. 10/413,779 assigned to the assignee of the present invention entitled, “RANDOM NUMBER GENERATOR METHOD AND SYSTEM FOR GENETIC ALGORITHM ANALYSIS” by Shackleford and Tanaka.

In one implementation, two of the chromosomes are selected at random from among the chromosomes in the population memory as a pair of parent chromosomes (one for each parent) (306). The corresponding fitness value from each new parent is compared with the fitness value of the resultant child chromosome. If the comparison indicates the fitness value of the newly created child chromosome is more fit, than the least-fit parent chromosome then the child chromosome replaces the least-fit parent chromosome in the population memory.

A probabilistic crossover operation between the first and second parent chromosomes produces a child chromosome (308). One or more randomly selected cut points on the pair of chromosomes delineate the sections of the parent chromosome to be used in the creation of the child chromosome. Both parent chromosomes are cut at the same cut point(s) and combined together to create the new child chromosome. For example, a single cut point produces a child chromosome composed of left-cut portion of a first parent chromosome and the right-cut portion of a second parent chromosome.

While one implementation of the present invention uses a single cut-point, it is also possible that multiple cut-points are selected and used in creating the child chromosome. Further, it is also possible that no cut-point is selected in which case one parent chromosome is copied and used directly to create the new child chromosome. It should be appreciated that both location of the cut-point(s) and the decision to perform the cross-over occur probabilistically and are not predetermined.

The resultant child chromosome is also mutated through a probabilistic alteration of the bits representing the child chromosome (310). In one implementation, a low-probability of 1 per-cent per bit is selected as the likelihood that a bit value will be mutated into another bit value. All bits have the same independent chance of mutation, so multiple bit changes in an n-bit chromosome are possible but less likely than a single-bit mutation. Typically, each bit in the child chromosome is mutated by inverting 0s to 1s and vice versa.

After the mutation operation, the child chromosome is evaluated and processed by a fitness function (312). Each fitness function is designed to solve different problems within the GA analysis framework and can be implemented in software, hardware, firmware, combinations thereof, and may include Very Large Scale Integration (VLSI) circuit or Field Programmable Gate Array (FPGA) technologies, for example. To solve a new problem, a different fitness function can be designed and implemented within substantially the same GA analysis framework described herein. The fitness function processes the child chromosome and produces a fitness value indicating of how well the particular child chromosome solves the given problem. For example, a child chromosome solves a proteomic problem by suggesting a combination of amino acids determined to correspond to a particular protein being analyzed or researched.

The child chromosome and the corresponding fitness value are used to determine whether the child chromosome survives and potentially replaces a parent chromosome in the population memory (314). The fitness value associated with the child chromosome is compared with the fitness value corresponding to the least fit parent chromosome in the current population memory to determine if the child chromosome survives. If the survival comparison indicates the child chromosome is more fit than the least-fit parent chromosome, the child chromosome replaces the chromosome in the population memory corresponding to the least-fit parent chromosome. Typically, the child electronic chromosome is considered more-fit if the fitness value indicates the elements in the child electronic chromosome more closely correspond to the compound being analyzed or studied. By repeating this process, the solution quality of the problem being solved by the GA increases as well as the overall fitness of the population.

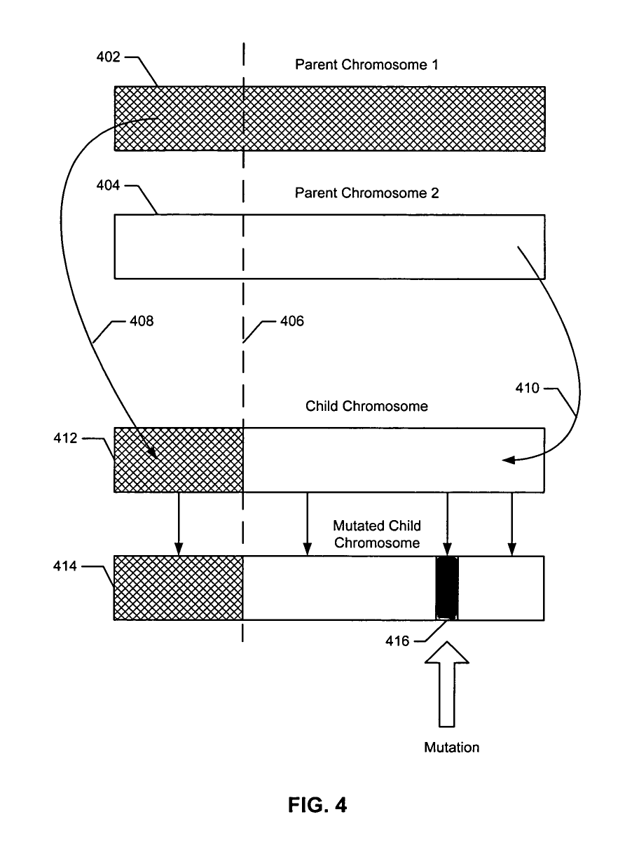

FIG. 4 is a block diagram illustrating both the cross-over operation between parent chromosomes and the mutation operation on a child chromosome. In this example, parent chromosome 402 and parent chromosome 404 are split along a single cut-point 406. Each parent contributes through cross-over operation 408 and cross-over operation 410 a portion of their electronic chromosome based on cut-point 406.

A child chromosome 412 having characteristics of both parent chromosomes is produced by these cross-over operations. Because the cut-point location is determined randomly, child chromosome 412 may have different proportions of each parent chromosome and is not limited to the combination illustrated herein. Multiple cut-points could also be used resulting in different portions of chromosomes from the parent chromosomes.

A mutation operation applied bit-wise to child chromosome 412 causes a probabilistic variation in binary representation of child chromosome 412. Although the probability of mutation is often low, the mutation helps explore other potential solutions or combinations that may not have existed or been available in the existing population memory. Mutation assists in rapid convergence on an optimal solution without testing every possible combination. In a protein sequencing problem for example, a mutation replaces a subfield of the child chromosome corresponding to one amino acid with another amino acid that may more closely solve the protein sequencing problem at hand.

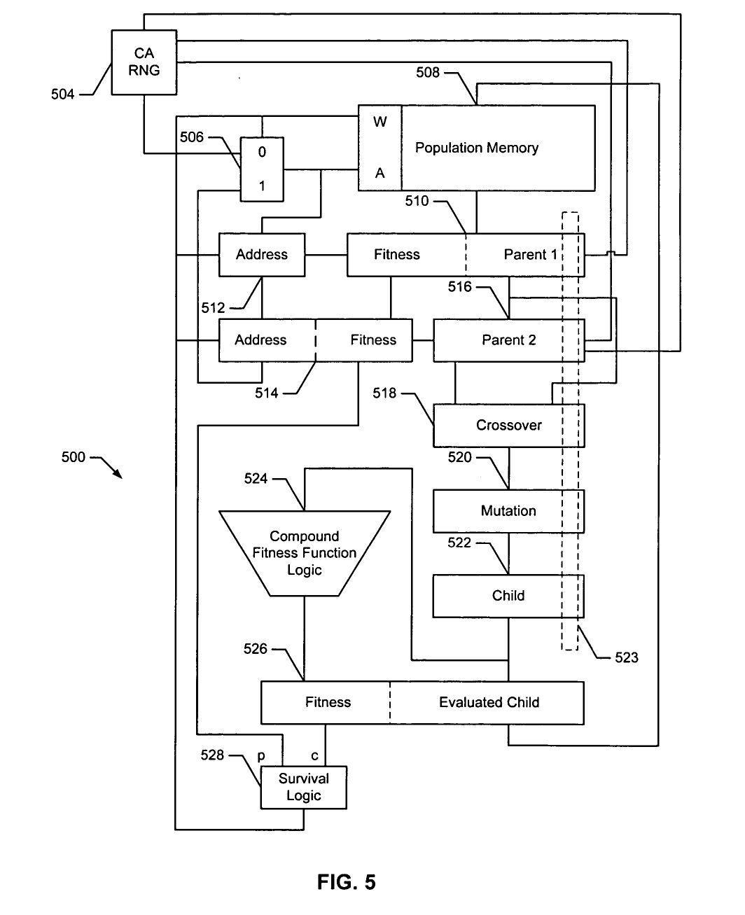

FIG. 5 is block diagram representation of a GA circuit 500 designed in accordance with one implementation of the present invention. GA circuit 500 includes a cellular automata random number generator (CA RNG) 504, a population memory MUX 506, a population memory 508, a parent 1 and fitness register 510, a parent 1 address register 512, a parent 2 address and fitness register 514, a parent 2 register 516, crossover logic 518, mutation logic 520, child register 522, a bit-slice GA/ RNG 523, compound fitness function logic 524, evaluated child and fitness register 526, and survival logic 528.

During initialization mode, CA RNG 504 produces random numbers used for a variety of purposes in the GA circuitry other than loading population memory 508 with the population chromosomes. For example, CA RNG 504 can be used effectively to generate random addresses for selecting parents as well as random data to produce random crossover and mutation operations in the GA circuitry. In general, CA RNG 504 can be used for generating random numbers in the GA circuitry where scalability is not required and a bit-slice based GA/RNG is not necessary or justified. MUX 506 is used to either select a parent using a random address generated by CA RNG 504 or it is used to replace a less-fit parent chromosome with a more-fit child chromosome by using the address portion of the register 514. When scalability is desired, bit-slice GA/ RNG 523 is used to generate random numbers for an n-bit wide chromosomes in population memory 508. Details on bit-slice GA/RNG 523 are described in further detail in U.S. patent application Ser. No. 10/413,779 assigned to the assignee of the present invention entitled, “RANDOM NUMBER GENERATOR METHOD AND SYSTEM FOR GENETIC ALGORITHM” by Shackleford and Tanaka.

In running or operating mode, the genetic analysis circuit randomly selects from population memory 508 a chromosome, fitness value, and address for the first parent. These values are loaded into parent 1 and fitness register 510 and parent 1 address register 512. The genetic analysis circuit also randomly selects from population memory 508 a chromosome, fitness value, and address for the second parent chromosome and then loads them into parent 2 address and fitness register 514, and parent 2 register 516. To save clock cycles spent accessing memory, the contents of the register associated with parent 1 can also be shifted into parent 2 for subsequent iterations rather than loading parent 2 directly from memory.

Crossover logic 518 combines the parent 1 and parent 2 values in a probabilistic manner as previously described. Mutation of the resulting combination between parent 1 and parent 2 occurs, if at all, in mutation logic 520 and then is stored in child register 522 for further processing.

Each new child chromosome in child register 522 is also provided with a fitness value. Fitness function logic 524 processes the child chromosome stored in child register 522 according to the predetermined evaluation criteria to create the initial fitness value. This fitness value for the child chromosome is stored along with the child chromosome in evaluated child and fitness register 526 awaiting further processing/evaluation. In one implementation, the child fitness value in evaluated child and fitness register 526 and compared with the corresponding fitness of the lesser fit parent in population memory 508 based using survival logic 528. To locate the lesser fit parent more readily, the address of the lesser fit parent can be stored in a lesser-fit register or cache. If the child chromosome has a better fitness than the lesser fit parent, it replaces the lesser fit parent and is stored at the lesser fit parent's address in population memory 508. Over time, the random numbers generated by an implementation of the present invention evolve into an optimal solution in accordance with the present invention and according to the GA analysis process.

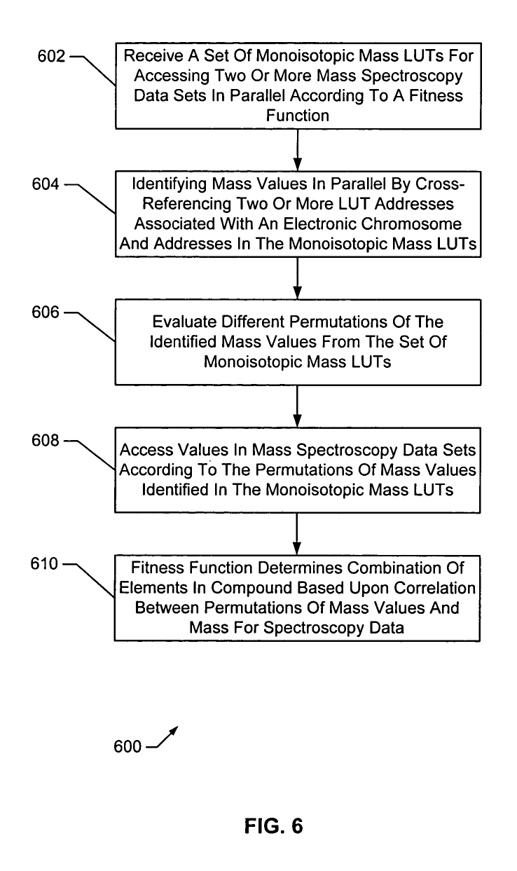

FIG. 6 is a flow chart diagram of the operations for using a fitness function in accordance with the present invention. An alternate implementation of the present invention concerns operation of the fitness function with reduced memory or storage requirements and is described in co-pending U.S. patent application Ser. No. 10/618,189 assigned to the assignee of the present invention entitled, “STORAGE REDUCTION METHOD AND APPARATUS FOR MASS SPECTROSCOPY ANALYSIS” by Shackleford and Tanaka.

Initially, a fitness function receives a set of monoisotopic mass look-up-tables (LUTs) for accessing and analyzing data sets in parallel (602). The LUTs associate LUT addresses with different mass values for a set of elements that potentially make up the compound being studied. For example, the LUTs are filled with mass values of different amino acids and a binary address for addressing these mass values as illustrated in FIG. 2.

In one implementation, the fitness function identifies mass values in parallel by cross-referencing two or more LUT addresses associated with an electronic chromosome and addresses in the monoisotopic mass LUTs (604). The number of mass values processed in parallel depends on the number of addresses specified in the electronic chromosome. For example, an electronic chromosome having six addresses allows the fitness function to explore permutations of one to six different amino acids or elements for a particular protein or compound. The fitness function converts addresses into mass values by way of the monoisotopic mass LUTs; these mass values are then used for analysis.

For example, the sum of six different monoisotopic masses may be used to address an entry in a mass spectroscopy table. The mass spectroscopy table includes the mass/charge and relative intensity information gathered through mass spectroscopy processing of the particular protein or compound being studied as illustrated in FIG. 1B. Thus, accessing the mass spectroscopy data set values depends on the combined mass values at the offset in one or more monoisotopic mass LUTs.

Fitness function evaluates different permutations of the identified mass values from the set of monoisotopic mass LUTs (606). In one implementation, the fitness function may add or subtract constant values to the various mass values and add or subtract the mass values together as deemed appropriate for the protein or compound being analyzed. If the compound being studied is a protein, the potential elements used in the protein sequencing analysis include one or more amino acids selected from a set of amino acids including: Gly, Ala, Ser, Pro, Val, Thr, Cys, Ile, Leu, Asn, Asp, Gln, Glu, Met, His, Phe, Arg, Tyr, Trp and Lys. Alternate components other than amino acids may also be used depending on the branch of research performed. These other research areas include combinatorial chemistry, medicinal chemistry, high-throughput screening and genomics.

The fitness function then accesses values in mass spectroscopy data sets according to the permutations of mass values identified in the monoisotopic mass LUTs (608). A higher fitness function value results when a given permutation of mass values matches or substantially matches a mass value measured and stored in the mass spectroscopy data set. Similarly, if a mass value does not match the spectroscopy data set then a lower fitness function value results.

The fitness function determines the combination of elements in the compound according to the correlation between the permutations of mass values and the mass values associated with the mass spectroscopy data set (610). A relatively large value produced by the fitness function indicates that the combination of elements is close to the sample mass. Likewise, a relatively small value is produced by the fitness function when the theoretical combination of elements is not close.

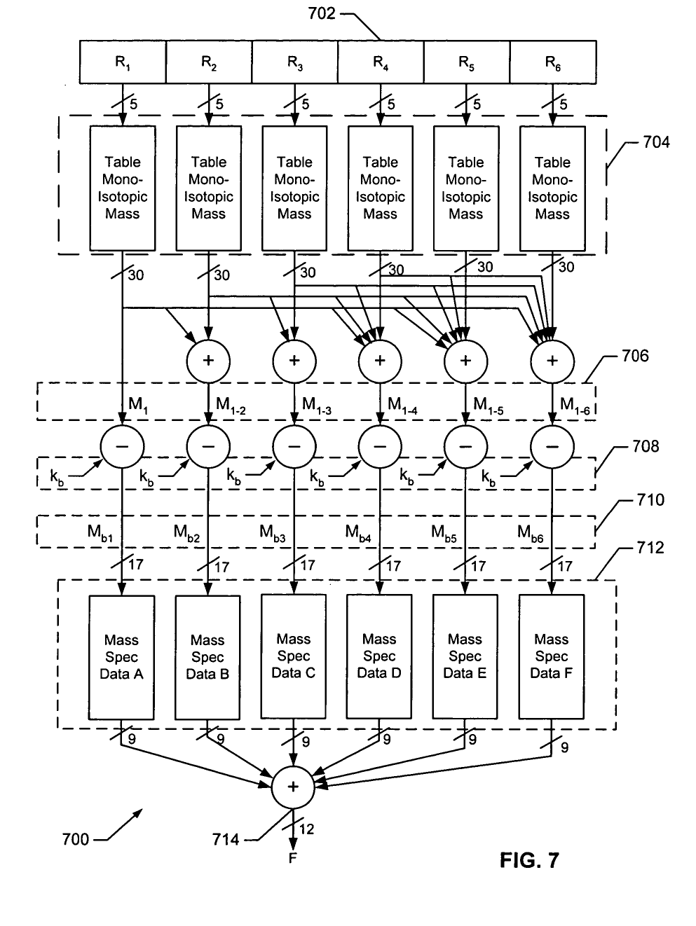

FIG. 7 is a block diagram illustration of a fitness function 700 implemented in hardware and used to identify an amino acid sequence for a protein. In this example, fitness function 700 includes a chromosome register 702, monoisotopic mass look-up-tables (LUTs) 704, a set of reside masses 706, a set of constants (k_{b}) 708 for adjusting the residue mass value, a set of b-ion reside masses 710 and mass spectroscopy data 712 collected from the mass spectroscopy device for a sample protein. Alternative, fitness functions could also be created to study other compounds or areas of scientific research.

In operation, chromosome register 702 is loaded with a hypothetical set of six amino acids also identified as residue values. In one implementation, the initial representation of amino acids loaded in the chromosome register 702 are generated in accordance with Genetic Algorithm (GA) principles as further described in co-pending United States Patent Application entitled, “RANDOM NUMBER GENERATOR METHOD AND SYSTEM FOR GENETIC ALGORITHM ANALYSIS”, Ser. No. 10/413,779 by J. Barry Shackleford and Motoo Tanaka assigned to the assignee of the present invention.

Initially, GA logic generates a random sequence of amino acids and iteratively evaluates the amino acid according to a fitness function. Eventually, the GA logic converges upon an optimal solution or sequence of amino acids. An alternate solution to using GA, loads every possible combination and sequence amino acids into chromosome register 702 and selects the sequence that best fits according to the fitness function. This latter “brute force” approach may take more time to process as each possible sequence entered into chromosome register 702 must be determined and evaluated. In contrast, GA analysis is likely to converge on the solution more rapidly but requires additional hardware as described in further detail later herein.

The 20 amino acids are represented by 19different mass values as two of the amino acids have essentially the same mass. Accordingly, each of the six residues in chromosome register 702 stores a 5-bit value identifying one of the 19 different mass values. The selected 5-bit identifier addresses a mass value from monoisotopic mass LUTs 704 and outputs a 30-bit number corresponding to the mass value. The mass value selected from monoisotopic mass LUTs 704 is processed according to logic within fitness function 700 as illustrated creating various permutations of the mass values.

In this example, fitness function 700 adds one or more of the six mass values selected from monoisotopic mass LUTs 704 together in different combinations to generate residue masses 706. The set of constants 708 are subtracted from the set of residue masses 706 creating the set of b-ion residue mass 710 to be matched with mass spectroscopy data 712. If the hypothetical amino acid sequence loaded in chromosome register 702 matches the mass measurement associated with the protein then a large fitness function value 714 is produced indicating that the combination of elements in the protein compound have been identified.

By analyzing operation of fitness function 700 in FIG. 7, it is observed that several of the mass spectroscopy data sets do not need to store 2¹⁷ or 131,072 different potential mass values collected by the mass spectrometer. Accordingly, these excess storage areas can be eliminated to save storage requirements and costs without impacting performance. Details on reducing the storage requirements for implementing the present invention are described in co-pending patent application Ser. No. 10/618,189 assigned to the assignee of the present invention, entitled “STORAGE REDUCTION METHOD AND APPARATUS FOR MASS SPECTROSCOPY ANALYSIS”, by Shackleford and Tanaka.

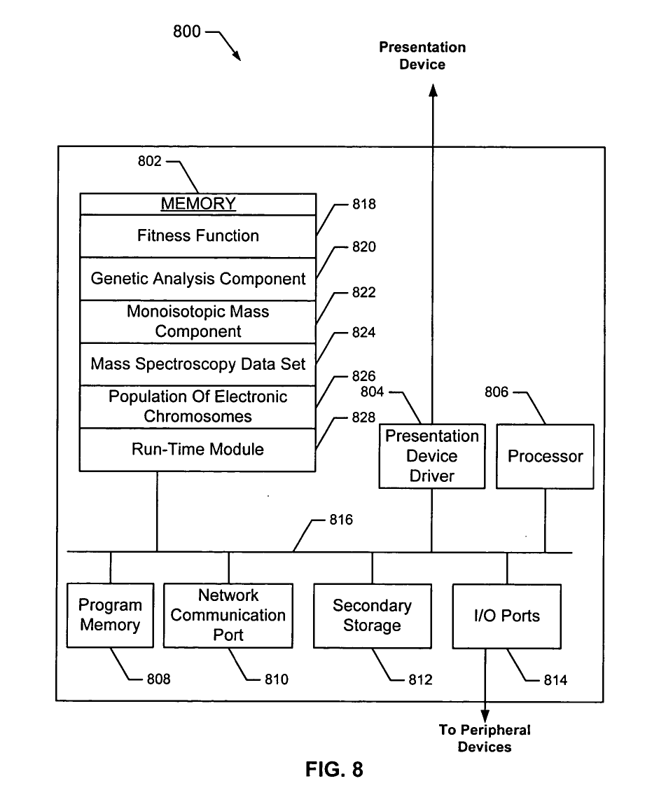

FIG. 8 is a block diagram of a system 800 used in one implementation for performing the apparatus or methods of the present invention. System 800 includes a memory 802 to hold executing programs (typically random access memory (RAM) or read-only memory (ROM) such as a flash ROM), a presentation device driver 804 capable of interfacing and driving a display or output device, a processor 806, a program memory 808 for holding drivers or other frequently used programs, a network communication port 810 for data communication, a secondary storage 812 with secondary storage controller, and input/output (I/O) ports 814 also with I/O controller operatively coupled together over a bus 816. The system 800 can be preprogrammed, in ROM, for example, using field-programmable gate array (FPGA) technology or it can be programmed (and reprogrammed) by loading a program from another source (for example, from a floppy disk, a CD-ROM, or another computer). Also, system 800 can be implemented using customized application specific integrated circuits (ASICs).

In one implementation, memory 802 includes a fitness function 818, a Genetic analysis component 820, a monoisotopic mass component 822, a mass spectroscopy data set 824 and a population of electronic chromosomes 826. Implementations of the present invention use a run-time module 826 to manage system resources used when processing one or more of the above components on system 800.

As previously described, fitness function 818 is designed to solve a particular problem using GA. In the previously described example, the fitness function uses amino acids in solving a protein sequencing problem however implementations of the present invention could also use different fitness functions and solve many different problems. Genetic analysis component 820 performs the genetic analysis algorithms as previously described to more rapidly converge upon a solution.

Monoisotopic mass component 822 provides a list of possible masses to be combined together when identifying a compound. These combinations of masses are compared with entries in the mass spectroscopy data set.

Mass spectroscopy data set 824 includes data produced when a protein is processed by a mass spectroscopy device. This includes the mass-charge (m/z) measurements along with relative intensity levels for determining both a sequence and quantity of elements in the compound.

Population of Electronic chromosomes 826 is a collection of electronic chromosomes used to identify the most likely combination of elements in a compound being analyzed. When identifying proteins, each electronic chromosome in population of electronic chromosomes 826 contains a combination of amino acids that may match up with the protein being studied.

While examples and implementations have been described, they should not serve to limit any aspect of the present invention. Accordingly, implementations of the invention can be implemented in digital electronic circuitry, or in computer hardware, firmware, software, or in combinations of them. Apparatus of the invention can be implemented in a computer program product tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs.

Further, while specific embodiments have been described herein for purposes of illustration, various modifications may be made without departing from the spirit and scope of the invention. For example, implementations of the present invention describe identifying amino acid combinations using a fitness function however alternate implementations can also use a fitness function and genetic algorithm hardware or software in analyzing and identifying many other compounds found in areas of biology, biotechnology, combinatorial chemistry, medicinal chemistry, high throughput screening and genomics. This includes, but is not limited to, the study of proteins or proteomics in addition to the broader range of materials found in combinatorial chemistry and compounds. Genetic analysis or GA and a properly formulated fitness function can be used greatly improve the task of performing research and discovering new functions. Accordingly, the invention is not limited to the above-described implementations, but instead is defined by the appended claims in light of their full scope of equivalents.

1. A method of discovering the elements in a compound with a fitness function, comprising:
receiving a set of monoisotopic mass look-up-tables (LUTs) that associates LUT addresses with mass values for a set of elements;
identifying mass values in parallel by cross-referencing two or more LUT addresses associated with an electronic chromosome and addresses in the monoisotopic mass LUTs;
evaluating different permutations of the identified mass values from the set of monoisotopic mass LUTs;
accessing values in two or more mass spectroscopy data sets according to the permutations of mass values identified in the monoisotopic mass LUTs; and
determining the combination of elements in the compound according to a correlation between the permutations of mass values and the mass values associated with the mass spectroscopy data set.

2. The method of claim 1 wherein evaluating different permutations further comprises:
combining one or more mass values from the set of monoisotopic mass LUTs together; and
adding one or more constant values to the combination of one or more mass values.

3. The method of claim 1 wherein the compound is a protein and the elements include one or more amino acids to be discovered during protein sequencing analysis.

4. The method of claim 2 wherein the one or more amino acids are selected from a set of amino acids including: Gly, Ala, Ser, Pro, Val, Thr, Cys, Ile, Leu, Asn, Asp, Gln, Glu, Met, His, Phe, Arg, Tyr, Trp and Lys.

5. The method of claim 1 wherein discovering elements of a compound relates to aspects of combinatorial chemistry and genomics.

6. The method of claim 1 wherein discovering elements of a compound relates to aspects of medicinal chemistry and high-throughput screening used in conjunction discovering medicinal chemical compounds.

7. The method of claim 1 wherein evaluating different permutations may include one or more of the following: adding or subtracting constants values, summing one or more different mass values.

8. The method of claim 1 wherein the electronic chromosome is a storage area with a series of addresses corresponding to mass values and potentially the elements in the compound.

9. A method of discovering elements of a compound using genetic algorithm computation, comprising:
receiving a population of electronic chromosomes each representing a set of elements and having a fitness value determined using a fitness function indicating the probability that at least one of the electronic chromosome provides the elements in the compound;
selecting at least two electronic chromosomes randomly from the population of electronic chromosomes as parent electronic chromosomes;
creating a child electronic chromosome through a crossover of the at least two parent electronic chromosomes using one or more randomly selected cut-points on the parent electronic chromosomes; and
evaluating the fitness value of the resulting child electronic chromosome using the fitness function to determine if elements in the child electronic chromosome correspond to the compound.

10. The method of claim 9, further comprising:
determining if the child electronic chromosome enters the population of electronic chromosomes when the fitness value indicates that the elements associated with the child electronic chromosome more likely represents the elements in the compound than the an electronic chromosome already in the population of electronic chromosomes.

11. The method of claim 9 further comprising:
mutating a child electronic chromosome through a probabilistic alteration of the bits representing the child electronic chromosome and a potential combination of elements in the compound.

12. The method of claim 9 wherein the compound is a protein and the elements include one or more amino acids to be discovered during protein sequencing analysis.

13. The method of claim 12 wherein the one or more amino acids are selected from a set of amino acids including: Gly, Ala, Ser, Pro, Val, Thr, Cys, Ile, Leu, Asn, Asp, Gln, Glu, Met, His, Phe, Arg, Tyr, Trp and Lys.

14. The method of claim 9 wherein discovering elements of a compound relates to aspects of combinatorial chemistry and genomics.

15. The method of claim 9 wherein discovering elements of a compound relates to aspects of medicinal chemistry and high-throughput screening used in conjunction discovering medicinal chemical compounds.

16. The method of claim 9 wherein the electronic chromosome is a storage area with a series of addresses corresponding to mass values and potential elements in the compound.

17. The method of claim 9 wherein the fitness function, comprises receiving a set of monoisotopic mass look-up-tables (LUTs) that associates LUT addresses with mass values for a set of elements;
identifying mass values in parallel by cross-referencing two or more LUT addresses associated with an electronic chromosome and addresses in the monoisotopic mass LUTs;
evaluating different permutations of the identified mass values from the set of monoisotopic mass LUTs;
accessing values in two or more mass spectroscopy data sets according to the permutations of mass values identified in the LUT; and
determining the combination of elements in the compound according to a correlation between the permutations of mass values and the mass values associated with the mass spectroscopy data set.

18. A computer program product for discovering the elements in a compound with a fitness function, tangibly stored on a computer-readable medium, comprising instructions operable to cause a programmable processor to:
receive a set of monoisotopic mass look-up-tables (LUTs) that associates LUT addresses with mass values for a set of elements;
identify mass values in parallel by cross-referencing two or more LUT addresses associated with an electronic chromosome and addresses in the monoisotopic mass LUTs;
evaluate different permutations of the identified mass values from the set of monoisotopic mass LUTs;
access values in two or more mass spectroscopy data sets according to the permutations of mass values identified in the monoisotopic mass LUTs; and
determine the combination of elements in the compound according to a correlation between the permutations of mass values and the mass values associated with the mass spectroscopy data set.

19. The computer program product of claim 18 wherein the compound is a protein and the elements include one or more amino acids to be discovered during protein sequencing analysis.

20. The computer program product of claim 18 wherein evaluating different permutations may include one or more of the following: adding or subtracting constants values, summing one or more different mass values.

21. A computer program product for discovering elements of a compound using genetic algorithm computation, tangibly stored on a computer-readable medium, comprising instructions operable to cause a programmable processor to:
receive a population of electronic chromosomes each representing a set of elements and having a fitness value determined using a fitness function indicating the probability that at least one of the electronic chromosome provides the elements in the compound;
select at least two electronic chromosomes randomly from the population of electronic chromosomes as parent electronic chromosomes;
create a child electronic chromosome through a crossover of the at least two parent electronic chromosomes using one or more randomly selected cut-points on the parent electronic chromosomes; and
evaluate the fitness value of the resulting child electronic chromosome using the fitness function to determine if elements in the child electronic chromosome correspond to the compound.

22. The computer program product of claim 21, further comprising instructions to:
determine if the child electronic chromosome enters the population of electronic chromosomes when the fitness value indicates that the elements associated with the child electronic chromosome more likely represents the elements in the compound than the an electronic chromosome already in the population of electronic chromosomes.

23. The computer program product of claim 21, further comprising instructions to:
mutate a child electronic chromosome through a probabilistic alteration of the bits representing the child electronic chromosome and a potential combination of elements in the compound.

24. The computer program product of claim 21, wherein the compound is a protein and the elements include one or more amino acids to be discovered during protein sequencing analysis.

25. An apparatus for discovering the elements in a compound with a fitness function, comprising:
a set of monoisotopic mass look-up-tables (LUTs) that associates LUT addresses with mass values for a set of elements;
a set of registers operatively connected to the set of monoisotopic mass LUTs that identifies mass values in parallel by cross-referencing two or more LUT addresses associated with an electronic chromosome and addresses in the monoisotopic mass LUTs;
arithmetic logic that evaluates different permutations of the identified mass values from the set of monoisotopic mass LUTs;
storage driven by arithmetic logic to access values in two or more mass spectroscopy data sets according to the permutations of mass values identified in the monoisotopic mass LUTs; and
arithmetic logic that determines the combination of elements in the compound according to a correlation between the permutations of mass values and the mass values associated with the mass spectroscopy data set.

26. An apparatus for discovering elements of a compound using genetic algorithm computation, comprising:
a population of electronic chromosomes in storage each representing a set of elements and having a fitness value determined using a fitness function that indicates the probability that at least one of the electronic chromosome provides the elements in the compound;
selection logic operatively coupled to the population of electronic chromosomes in storage that selects at least two electronic chromosomes randomly from the population of electronic chromosomes as parent electronic chromosomes;
a child electronic chromosome register that receives a crossover of the two parent electronic chromosomes using one or more randomly selected cut-points on the parent electronic chromosomes; and
compound fitness function logic that evaluates the fitness value of the resulting child electronic chromosome using the fitness function to determine if elements in the child electronic chromosome correspond to the compound.

27. An apparatus for discovering the elements in a compound with a fitness function, comprising:
means for receiving a set of monoisotopic mass look-up-tables (LUTs) that associates LUT addresses with mass values for a set of elements;
means for identifying mass values in parallel by cross-referencing two or more LUT addresses associated with an electronic chromosome and addresses in the monoisotopic mass LUTs;
means for evaluating different permutations of the identified mass values from the set of monoisotopic mass LUTs;
means for accessing values in two or more mass spectroscopy data sets according to the permutations of mass values identified in the monoisotopic mass LUTs; and
means for determining the combination of elements in the compound according to a correlation between the permutations of mass values and the mass values associated with the mass spectroscopy data set.

28. An apparatus for discovering elements of a compound using genetic algorithm computation, comprising:
means for receiving a population of electronic chromosomes each representing a set of elements and having a fitness value determined using a fitness function indicating the probability that at least one of the electronic chromosome provides the elements in the compound;
means for selecting at least two electronic chromosomes randomly from the population of electronic chromosomes as parent electronic chromosomes;
means for creating a child electronic chromosome through a crossover of the at least two parent electronic chromosomes using one or more randomly selected cut-points on the parent electronic chromosomes; and
means for evaluating the fitness value of the resulting child electronic chromosome using the fitness function to determine if elements in the child electronic chromosome correspond to the compound.
